(19) United States
(12) Patent Application Publication
(10) Pub. No.: US 2005/0112740 A1
(43) Pub. Date: May 26, 2005

(54) Waste metals recycling-methods, processed and systems for the recycle of metals into coagulants

(76) Inventor: Richard Alan Haase, Missouri City, TX (US)

Correspondence Address: RICHARD A. HAASE (INVENTOR), 4402 RINGROSE DRIVE, MISSOURI CITY, TX, 77459, US

(21) Appl. No.: 10/969,393

(22) Filed: Oct. 20, 2004

Related U.S. Application Data
(60) Provisional application No. 60/512,839, filed on Oct. 20, 2003.

(51) Int. Cl. C12P 3/00; C12N 1/21

(52) U.S. Cl. 435/168; 435/252.3

(57) ABSTRACT

This invention presents chemical and biological methods, processes and systems for purifying, reclaiming and/or recycling metal(s) in aqueous solution. This invention presents methods, processes and systems for purifying, reclaiming and/or recycling: waste sludge from water purification plants, waste catalyst form polymer manufacturing plants and other waste aqueous metal streams, wherein said waste stream(s) contains at least one metal in concert with BOD and/or TOC and/or COD. Removal of at least one of: BOD, TOC, COD and any combination therein is accomplished via a biological reactor, wherein it is most preferred that an operating pH of 9.25+/−0.50 is maintained to maximize the insoluble oxide and/or hydroxide form of the metal, while minimizing the ionic form, toxic form, of the metal, thereby providing an environment which is conducive to biological activity. Post biological reaction, metal(s) are removed from aqueous solution with liquid/solids separation. Post biological reaction bacteria are removed from aqueous solution with liquid/solids separation. In the most preferred embodiment, the metals from liquid/solids separation are recycled into coagulant manufacture.

### RELATED APPLICATION DATA

This application claims priority based on a provisional application, 60/512,839 filed Oct. 20, 2003.

### FIELD OF THE INVENTION

This invention relates to chemical and biological methods, processes and systems for purifying, reclaiming and/or recycling metal(s) in aqueous solution. This invention relates to methods, processes and systems for purifying, reclaiming and/or recycling: waste sludge from water purification plants, waste catalyst form polymer manufacturing plants and other waste aqueous metal streams, wherein said waste stream(s) contains at least one metal in concert with BOD and/or TOC and/or COD. This invention relates to methods, processes and systems to recycle a waste metals(s) in order to purify, reclaim and/or recycle the metal as a coagulant. It is preferred that said recycled metal(s) is used in liquid solids separation, coagulation, to purify water. These metal coagulant(s) are applicable to the liquid solids separation, clarification, of any water having colloidal matter which contains a negative columbic charge. These coagulants are applicable to waste water, pool, pond, lake, drinking, industrial and process water purification. It is preferred that said recycled metal(s) be at least one of: aluminum, iron, magnesium, calcium and/or a combination thereof. It is most preferred that said recycled metal(s) be aluminum.

### DESCRIPTION OF THE RELATED ART

Municipal and industrial drinking water, industrial process water and wastewater treatment facilities must dispose of solids separated from water during treatment. The aqueous solution of these solids is often termed sludge; however, for clarity, the term aqueous separated solids (SS) is used, wherein said SS is primary solids and/or secondary solids. Primary solids are defined as aqueous solids that are separated from the treated water in primary treatment in any treatment system; wherein primary treatment physically separates aqueous solids from the treated water, usually in a clarifier, a dissolved air flotation device and/or a media type filter. Secondary solids, bio-solids, are defined as aqueous solids that are separated from treated water in secondary treatment; wherein secondary treatment is biological treatment, usually in a wastewater treatment plant. SS, as containing primary solids, are normally sent to dewatering and/or to digestion, prior to disposal. SS, as containing bio-solids, are normally sent to digestion, prior to disposal. In digestion, the solids volume is reduced by bacteria that consume, digest, the organic portion of SS. The performance of digestion is determined by the reduction of said organic portion, which is defined as Volatiles in the SS. Volatiles are defined in the laboratory as the solids remaining in a filter from a filtered sample after those filtered solids are heated to approximately 102° C., yet do not remain after a second heating to approximately 600° C. This mass measurement difference is a definition of the heavier organic content of the filter sample and is therefore an estimation of the biological content and organic biological food content of the solids in an aqueous sample. In mesophilic digestion, the percent Volatiles reduction is normally 40 to 50 percent. In thermophillic digestion, the percent Volatiles reduction can be 55 to 65 percent. Mesophiles are defined as bacteria that operate between temperatures of approximately 40 and 105° F. Thermophiles are defined as bacteria that operate between the temperatures of approximately 105 and 165° F. To manage transportation and disposal cost, nearly all wastewater treatment facilities prefer to reduce the Volatiles content of the digested solids, biological sludge, as much as is economically practical.

After digestion, the final digested solids product (Digested Solids, DS) must be properly disposed. Disposal of DS is normally accomplished by either land application or disposal in a landfill. To minimize the handling and transportation expense of DS, the water content of the DS is normally reduced from approximately 94 percent, in digestion, to approximately 75 percent by chemical and mechanical separation utilizing a belt press, centrifuge or other similar type dewatering device. To reduce the water content further, many facilities incorporate heated air-drying, evaporating, or any combination thereof with mechanical means.

Municipal raw drinking waters, and usually raw industrial waters, generally contain four types of human pathogenic organisms: bacteria, viruses, protozoa and helminthes (parasitic worms). In addition, municipal raw drinking water may contain organic contaminants, manmade or natural.

Municipal wastewaters, and usually industrial wastewaters, also generally contain four types of human organisms: bacteria, viruses, protozoa and helminthes (parasitic worms). The actual species and density of pathogens contained in raw wastewater will depend on the health of the particular community and/or the inclusion of significant rainwater runoff from animal sources. The level of pathogens contained in the untreated DS will depend on the flow scheme of the collection system and the type of wastewater treatment. For example, since pathogens are primarily associated with insoluble solids (non-volatile solids), untreated primary solids have a higher pathogen density than the incoming wastewater. This same physical phenomenon applies to the SS from drinking water purification.

One purpose of a water purification facility is to remove pathogens, bacteria, and viruses, from a water, as well as to protect against biological contamination reoccurring in the treated water. Said bacteria and/or viruses leave the facility in the wasted SS.

While the concentration of contaminants differs greatly from drinking water or industrial water clarification to wastewater clarification, the clarification process itself is rather similar. In both cases coagulants are used. In drinking water, to obtain the required treatment purity, metal coagulants are required. In wastewater, metal coagulants are sometimes used. In both cases, the coagulant is disposed of along with the sludge. In the case of drinking water sludge or SS, the valuable metal used in coagulation is both an environmental impact with disposal and an increased cost of operation, as the wasted metal coagulant in the sludge must be replaced in coagulation. This replacement often occurs from an aluminum or iron mine, creating considerable processing and transportation expense.

While it is most common for a wastewater treatment facility to have SS dewatering and/or handling equipment, it is not common for a drinking water facility to have SS dewatering equipment. It is very common for a drinking water facility to place the SS into a collection system, whereby the SS is transported to the wastewater treatment facility for treatment, along with other wastewater. It is very common for a drinking water facility to have a pond system, wherein water is decanted from the SS, prior to recycle of said water. This operating scenario is under scrutiny by the US EPA as the recycle of said decanted water has the potential to cycle up the pathogen concentration of the raw water in the drinking water plant. For those systems which have a pond separation system, the pond will be dredged when the level of SS is sufficiently high to affect separation or operation in the pond.

Metals are also used as catalysts in the production of organic polymers. Once the valence sites of the catalyst are spent, the catalyst is waste requiring disposal. This disposal, while being rich in the metal of catalysis, is expensive due to the environmental impact of the waste heavy metal(s) and in replacement of the catalyst.

TOC (Total Organic Carbon) is defined as the total amount of organic carbon in a water. By use if the term organic carbon, the definition of TOC is to be understood to refer to organic molecules and/or compounds, not free carbon or carbon salts. BOD (Biological Oxygen Demand) is defined as the oxygen required for ubiquitous bacteria to remove as much TOC as said ubiquitous bacteria are capable from a water; ubiquitous bacteria are bacteria which naturally occur in the environment. COD (Chemical Oxygen Demand) is defined as the amount of a known oxidizing chemical required to oxidize TOC within a water. COD differs from BOD in that COD includes a measure of the TOC which is not removable by ubiquitous bacteria. TOC differs from BOD in that TOC includes a measure of TOC which is not removable by ubiquitous bacteria. TOC differs from COD in that TOC includes an amount of TOC which cannot be oxidized by the oxidizer used to measure COD. Ubiquitous bacteria in a BOD analysis consume TOC from the water while using oxygen from the water, wherein much of said BOD could also measure as COD and/or as TOC; it is during this consumption that approximately a 1:1 relationship exists between each pound of BOD removed with each pound of oxygen required for biological removal. Therefore, BOD is an indirect measure of TOC in a water. Further, COD is an indirect measure of TOC in a water. Usually TOC≧COD≧BOD; the difference between each depends upon the composition of the organic molecules or compounds in a water.

Since pathogens only present a danger to humans and animals through physical contact, one important aspect in land application of SS or DS is to minimize, if not eliminate, the potential for pathogen transport. Minimization of pathogen transport is accomplished through reduction of Vector attraction. Vectors are any living organism capable of transmitting a pathogen from one organism to another either directly or indirectly by playing a key role in the life cycle of the pathogen. Vectors that are specifically related to SS or DS could most likely include birds, rodents and insects. The majority of vector attraction substances contained in the DS are in the form of Volatiles. If left unstabilized, Volatiles will degrade, produce odor and attract pathogen-carrying Vectors.

On Feb. 19, 1993, the National Sewage Use and Disposal Regulations (Chapter 40 Code of: Federal Regulations Part 503 and commonly referred to as the 503 Regulations) were published in the Federal Register. The 503 Regulations define DS treatment methods that transform DS into Class “A” DS: Class “A” DS is nominally free of pathogens and Vector attraction. In essence, the Regulation establishes several categories in terms of stabilization, pathogenic content, beneficial reuse and disposal practices for all land-applied DS. These regulations set forth chemical methods, temperature methods, methods that include a combination of chemical and temperature, as well as other methods, including composting to treat DS for land application. Since 1993, experience has taught that the most reliable methods of Vector reduction are the temperature methods and/or chemical methods. The temperature methods include direct heating and thermophillic digestion. The chemical methods include pH manipulation to either acidic and basic pH.

A thorough review of a water treatment facility can be obtained from many textbooks, which may include and are listed herein as references: “Water Supply and Pollution Control” by Clark et al., “Water Quality and Treament”, by the American Water Works Association, “Coagulation: by the American Water Works Association and “Optimizing Water Treatment Plant Performance Using the Composite Correction Program,” by the USEPA.

### SUMMARY OF THE INVENTION

A primary object of the invention is to devise an effective, efficient, and economically feasible process for recovering aluminum and/or iron from sludge and/or SS.

Another object of the invention is to devise an economically feasible process for recovering aluminum and/or iron from sludge and/or SS so that said aluminum and/or iron can be used as a coagulant in water purification.

Another object of the invention is to devise an economically feasible process for recovering aluminum and/or iron from waste catalyst streams.

Another object of the invention is to devise an economically feasible process for recovering aluminum and/or iron from waste catalyst streams so that said aluminum and/or iron can be used as a coagulant in water purification.

Another object of the invention is to devise an economically feasible process for reducing the sludge volume and/or recycling the metal coagulant(s) from a drinking water purification plant.

Additional objects and advantages of the invention will be set forth in part in a description which follows and in part will be obvious from the description, or may be learned by practice of the invention.

The bio-chemical pathway is, generally:

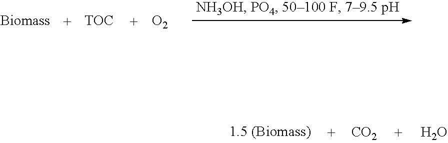

As long as the TOC is a consumable substrate, that is to say a consumable food source for bacteria biomass will consume TOC in direct proportion to the available biomass, oxygen, nutrients and available kinetics to bring the TOC in contact with the biomass. To that end, bacteria can consume BOD, TOC, and COD, depending on the type of bacteria employed, having the knowledge that ubiquitous bacteria will not consume substances which measure as TOC or COD after complete ubiquitous BOD removal. The above bio-chemical pathway removes BOD and/or TOC and/or COD by biological consumption of the BOD and/or TOC and/or COD (herein after referred to as simply TOC).

The bio-chemical pathway performs optimally at a pH of between about 7.0 and 9.0, and performs well between a pH of about 6.5 and about 9.5. The bio-chemical pathway performs, albeit at a lesser extent depending upon the type of the biological cultures between a pH of about 5.0 and 10.0.

Ammonia gas and ammonium hydroxide exist in water in an equilibrium, wherein said equilibrium is dependant upon pH. Ammonium hydroxide is a nutrient for TOC consuming bacteria and a food source for nitrifying bacteria. In all cases, ammonia gas is a biocide inhibiting and/or killing bacteria. Under conditions of ammonia-nitrogen less than approximately 150 mg/L, carbon consuming bacteria utilize ammonia-nitrogen as nutrient; and, nitrifying bacteria can remove ammonia-nitrogen, converting ammonia-nitrogen into nitrate and/or nitrite. Under conditions of ammonia-nitrogen greater than approximately 150 mg/L and less than approximately 350 mg/L, both bio-chemical pathways perform optimally at a pH of between approximately 7.0 and 8.0. At an ammonia-nitrogen of greater than about 350 mg/L at any pH, the concentration of ammonia gas in the water will at a minimum significantly inhibit biological activity.

Metals have a solubility that is directly related to pH. In acidic environments, below a pH of about 7.0, the concentration of a soluble metal(s) increases with lower pH. In basic environments, above a pH of about 10.5, the concentration of a soluble metal(s) increases with higher pH. Due to this pH/solubility relationship, metals are relatively amphoteric, with some metals having a greater amphoteric nature than others. Depending on the metal, the minimum soluble metal concentration in the water and the maximum concentration of said metal hydroxide and/or oxide exists at a pH of approximately 9.25+/−0.50. In hydroxide form, the insoluble metal can be separated from aqueous solution. In ion form, the soluble metal is difficult to separate from aqueous solution.

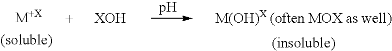

The instant invention has identified a method, process and system overlap in the pH range of the biological removal of BOD, TOC and COD (defined herein as simply TOC) with the maximum formation of insoluble metal hydroxides and/or oxides. The instant invention has found an operating scenario wherein the metal can be removed from aqueous solution in hydroxide and/or oxide form (herein after referred to as MOH) in combination with an operating scenario wherein a biological population can consume, remove. TOC from said solution. Said biological population can be mesophilic or thermophillic, aerobic or anaerobic. Said MOH is formed by pH.

The instant invention has identified an operating window, wherein an aqueous solution, SS, containing metal(s) in ionic form (herein after referred to as M(s)) and/or in a hydroxide/oxide form, the pH is increased and/or reduced to near 9.0+/−1.0, and preferably increased and/or reduced to near 9.25+/−0.50 to form an aqueous solution of MOH SS. TOC is removed from said MOH SS within a biological reactor. Once said SS containing said MOH is within said biological reactor, the bacteria within said biological reactor would consume the TOC, thereby creating an MOH solution (MOHS). Said biological reactor can be of any design as is known in the art. Said MOHS, after exiting said biological reactor would preferably be sent to a liquid/solid separation process. In said liquid/solid separation process, it is preferred that bacteria would be mostly separated from said MOH and water. In said liquid/solid separation process, it is preferred that said MOH would be mostly separated from said bacteria and water. Said water exiting said liquid/solid separation is to preferably be recycled to the process from which the SS stream came. Alternatively, said water could be sent to a wastewater treatment facility or disinfected and discharged. Said bacteria would be wasted as needed and/or sent back to the biological reactor. If said bacteria is wasted, it is preferred that said bacteria be digested and/or dewatered prior to disposal. If said bacteria is wasted, it is most preferred that said bacteria be converted to Class A material. Class A status is most preferably obtained with thermophillic digestion. Class A status is preferably obtained with any recognized method to obtain Class A status as is recognized by the US EPA 503 Regulations. Said MOH is preferably to be recycled in the metal industry. Said MOH is most preferably to be recycled in coagulant manufacture.

### BRIEF DESCRIPTION OF THE DRAWINGS

A better understanding of the present invention can be obtained when the following detailed description of the preferred embodiments are considered in conjunction with the following drawings, in which:

FIG. 1 illustrates in block diagram form a general description of the instant invention. In FIG. 1 all aqueous stream containing metal(s) and TOC is oxidized to a pH of preferably approximately 9.25+/−0.5. In the case wherein said aqueous stream has a pH above about 9.5 to 10.0, the pH is to be lowered with an acid. In the case wherein said aqueous stream has a pH below about 8.0 to 8.5, the pH is to be raised with a base. The pH adjusted aqueous stream flows into a biological reactor and/or biological reaction, wherein bacteria reduce said TOC. After biological reaction, the aqueous stream flows to a liquid-solid separation device, wherein at least one selected from a list consisting of: the MOH is mostly separate from the bacteria and the water, the bacteria is mostly separated from the MOH and the water, the water is mostly separated from the MOH and the bacteria, and any combination therein. It is preferred to recycle at least a portion of the bacteria back into the biological reactor to approach an activated sludge type of system, thereby to minimize equipment investment. Waste bio-solids are to be preferably digested. The waste bio-solids are most preferably to be thermophilic digested. The MOH is to be preferably recycled. The MOH is most preferably to be recycled into coagulant manufacture.

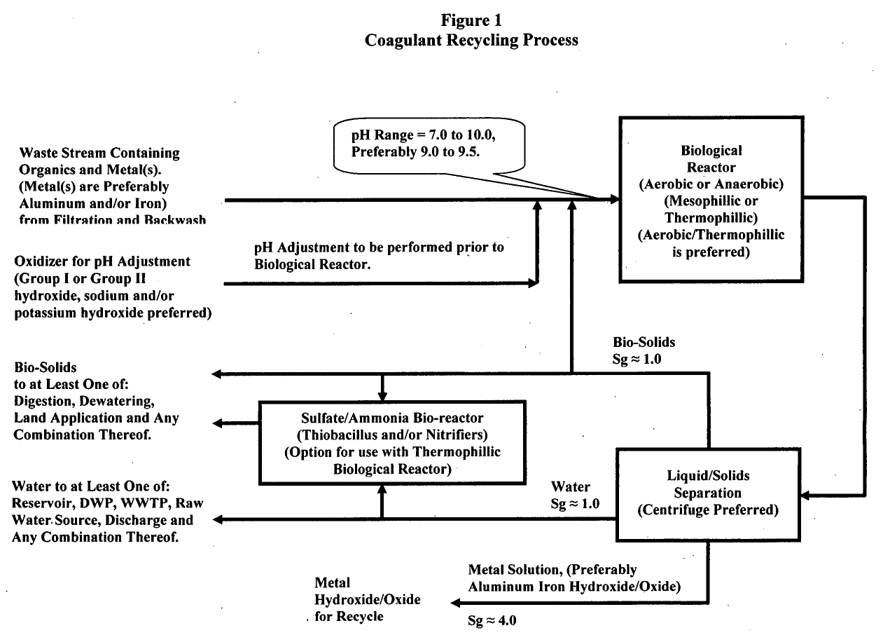

FIG. 2 illustrates in block diagram form a general description of the instant invention as it applies to a drinking water facility. In FIG. 2 an aqueous stream containing metal(s) and TOC is oxidized to a pH of preferably approximately 9.25+/−0.5. In the case wherein said aqueous stream has a pH above about 9.5 to 10.0, the pH is to be lowered with an acid. In the case wherein said aqueous stream has a pH below about 8.0 to 8.5, the pH is to be raised with a base. The pH adjusted aqueous stream flows into a biological reactor and/or biological reaction, wherein bacteria reduce said TOC. After biological reaction, the aqueous stream flows to a liquid-solid separation device, wherein at least one selected from a list consisting of: the MOH is mostly separated from the bacteria and the water, the bacteria is mostly separated from the MOH and the water, the water is mostly separated from the MOH and the bacteria, and any combination therein. It is preferred to recycle at least a portion of the bacteria back into the biological reactor to approach an activated sludge type of system, thereby to minimize equipment investment. Waste bio-solids are to be preferably digested. The waste bio-solids are most preferably to be thermophilic digested. The MOH is to be preferably recycled. The MOH is most preferably to be recycled into coagulant manufacture.

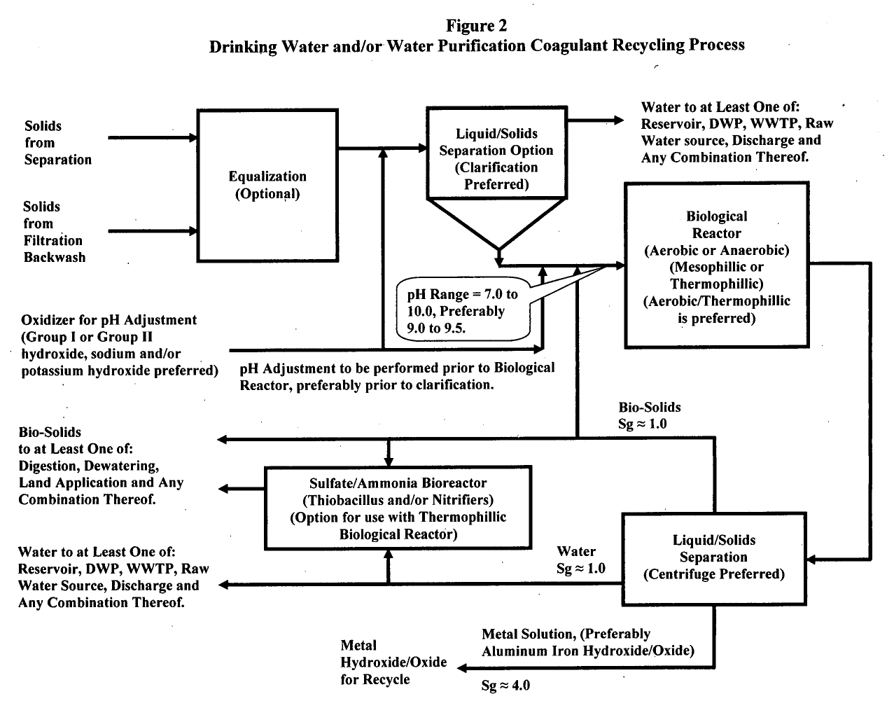

FIG. 3 illustrates in block diagram form a general description of the instant invention as it applies to a wastewater treatment facility. In FIG. 3 an aqueous stream containing metal(s) and TOC is oxidized to a pH of preferably approximately 9.25+/−0.5. In the case wherein said aqueous stream has a pH above about 9.5 to 10.0, the pH is to be lowered with an acid. In the case wherein said aqueous stream has a pH below about 8.0 to 8.5, the pH is to be raised with a base. The pH adjusted aqueous stream flows into a biological reactor and/or biological reaction, wherein bacteria reduce said TOC. After biological reaction, the aqueous stream flows to a liquid-solid separation device, wherein at least one selected from a list consisting of: the MOH is mostly separated from the bacteria and the water, the bacteria is mostly separated from the MOH and the water, the water is mostly separated from the MOH and the bacteria, and any combination therein. It is preferred to recycle at least a portion of the bacteria back into the biological reactor to approach an activated sludge type of system, thereby to minimize equipment investment. Waste bio-solids are to be preferably digested. The waste bio-solids are most preferably to be thermophilic digested. The MOH is to be preferably recycled. The MOH is most preferably to be recycled into coagulant manufacture.

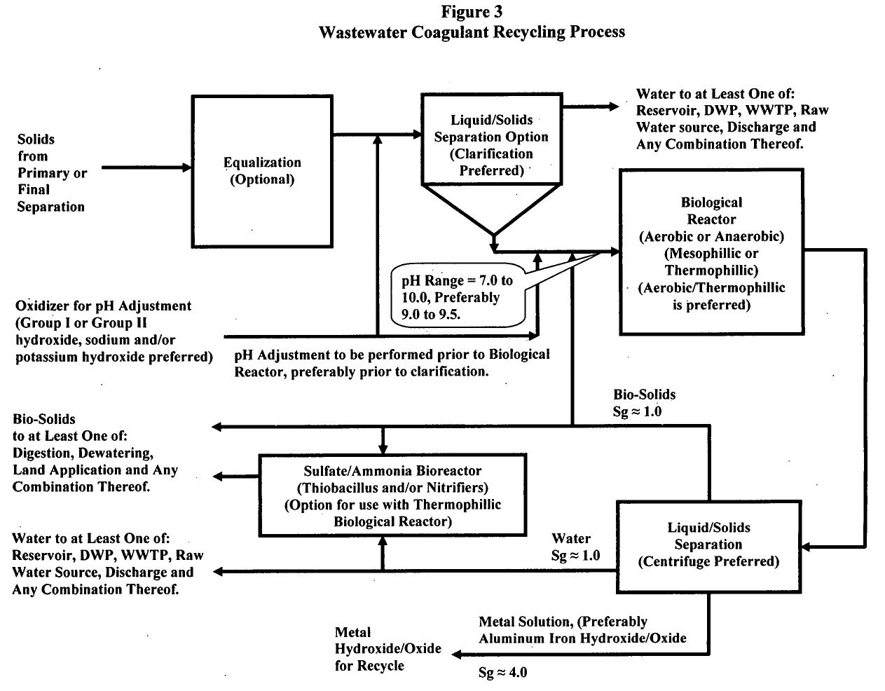

FIG. 4 illustrates in block diagram form a general description of the instant invention as it applies to a waste catalyst and/or to a waste metal(s) stream. In FIG. 4 an aqueous stream containing metal(s) and TOC is oxidized to a pH of preferably approximately 9.25+/−0.5. In the case wherein said aqueous stream has a pH above about 9.5 to 10.0, the pH is to be lowered with an acid. In the case wherein said aqueous stream has a pH below about 8.0 to 8.5, the pH is to be raised with a base. The pH adjusted aqueous stream flows into a biological reactor and/or biological reaction, wherein bacteria reduce said TOC. After biological reaction, the aqueous stream flows to a liquid-solid separation device, wherein at least one selected from a list consisting of: the MOH is mostly separated from the bacteria and the water, the bacteria is mostly separated from the MOH and the water, the water is mostly separated from the MOH and the bacteria, and any combination therein. It is preferred to recycle at least a portion of the bacteria back into the biological reactor to approach an activated sludge type of system, thereby to minimize equipment investment. Waste bio-solids are to be preferably digested. The waste bio-solids are most preferably to be thermophilic digested. The MOH is to be preferably, recycled. The MOH is most preferably to be recycled into coagulant manufacture.

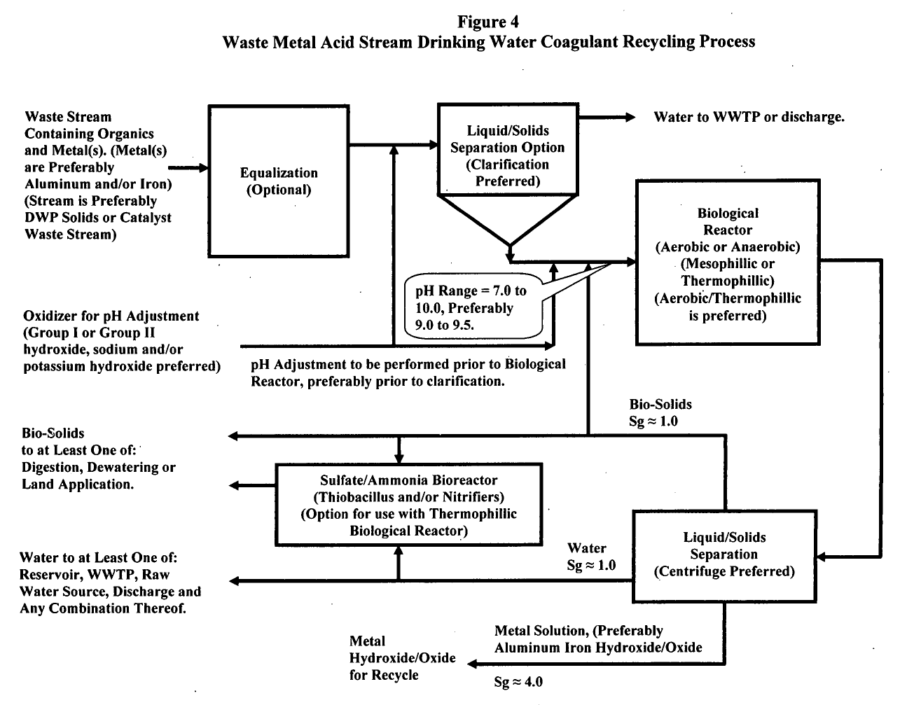

### DETAILED DESCRIPTION OF THE PREFERRED EMBODIMENTS

This instant invention describes methods, processes and systems for the separation and purification of a metal(s) in an aqueous solution containing metal(s). While the instant invention will perform well with most heavy metals, biological activity will depend on the toxicity of and the concentration of any heavy metal in the operating pH range. The operating pH range is preferably between about 8.0 and 10.0. The operating pH range is most preferably about 9.25+/−0.50 and is optimally about 9.25+/−0.25. The instant invention is most preferred to separate and purify metals used in liquid-solids separation. The most preferred method of liquid-solids separation is coagulation in the application of water clarification. The instant invention can be used to recycle any heavy metal, wherein the toxicity of the heavy metal does not significantly inhibit biological activity in the operating pH range.

Bacteria, bio-cultures, used in the biological reactor are preferably to be selected depending on the TOC, substrates. As is known in the art of biological augmentation, specific strains are known to break specific molecular bonds; this can be further classified into the breaking of specific molecular bonds and often further yet into the breaking of specific molecular bonds on specific substrates. For substrates which are toxic to ubiquitous bacteria, the bio-cultures used in the biological reactor are preferably to be selectively cultured on said toxic substrate. Selective culturing is the process of continuously providing a substrate to bacteria, either in concentrated form or blended with an easily consumable substrate, to a bio-culture, bacteria, or a blend of bacteria strains, usually in a laboratory environment, through many generations. After many generations, when the consumption of said toxic substrate is acceptable, the strain(s) are to be “Selectively cultured on that otherwise toxic substrate.” Often performance on a toxic substrate requires the addition of an easily consumable co-substrate to facilitate consumption of the toxic substrate. It is a preferred embodiment to provide a co-substrate to the biological reactor.

It is an embodiment that the bacteria in the biological reactor are ubiquitous. It is an embodiment to transport bio-mass, bacteria, to the biological reactor. It is a preferred embodiment that the bacteria utilized in the biological reactor be fermented of known bacteria strains. It is a most preferred embodiment that the bacteria utilized in the biological reactor be selectively cultured to specific substrate(s). It is also a most preferred embodiment that the bacteria utilized in the biological reactor be non-pathogenic. It is an optimally preferred embodiment that the bacteria utilized in the biological reactor be fermented of known bacteria strains, non-pathogenic and selectively cultured for known TOC substrates within the biological reactor. Preferably, only biological cultures, bacteria that are placed into the bacterial fermentation process are to be utilized in the biological reactor and/or biological reaction system. Preferred strains for inoculation in the biological reactor and/or biological reaction system comprise at least one selected from a list consisting of: Acinobacter, Nitrobacter, Enterobacter, Thiobacillus and Thiobacillus Denitrificanus, Psudomonas, Escherichia, Artobacter, Achromobacter, Bdellovibrio, Thiobacterium, Macromonas, Bacillus, Cornebacterium, Aeromonas, Alcaligenes, Falvobacterium, Vibro and fungi. Enzymes may be used; however, while enzymes increase short term biological effectiveness, enzymes tend to reduce the long term effectiveness of the biological cultures, thereby requiring continued use of enzymes. Therefore, enzymes are not preferred. In contrast, bacteria cultures which produce their own enzymatic activity are preferred. The above list is indicative of the strains that can be used; the list is not to be restrictive of the strains that can be used.

Bacteria operate per the Arrhenius equation in relation to temperature. Therefore, in addition to the above list of bacteria strains, alternate strains may be used for operation in different temperatures. While mesophiles operate between about 45 and 105° F., thermophiles operate between about 115 and 165 F, psychrotrophs operate between about −35 and 95° F., and psychrophiles operate between about −35 and 65° F. It is an embodiment that the biological reactor be messophilic and/or comprises mesophiles. It is an embodiment that the biological reactor be thermophilic and/or comprises thermophiles. It is an embodiment that the biological reactor comprises psychrotrophs. It is an embodiment that the biological reactor comprises psychrophiles. It is most preferred to utilize either psychrotrophs or psychrophiles in biological reaction for biological operating temperatures below about 50° F.

Whereas Thiobacillus and Thiobacillus Denitrificanus do not remove TOC, Thiobacillus and Thiobacillus Denitrificanus can remove sulfides, these species incorporate sulfur into their bio-mass similar to that of carbon for carbon consuming bacteria. Thiobacillus Denitrificanus, as well as many Denitrificanus species under low dissolved oxygen (DO) conditions (approximately <0.6 ppm DO), can also remove oxides of nitrogen, such as nitrous oxide, nitrite and nitrate. While sulfides present water with an objectionable odor, other TOC molecules, such as Geosmine and MIB, can present water with objectionable taste and odor. It is a preferred embodiment to provide blends of the above cultures with either Thiobacillus or Thiobacillus Denitrificanus to the biological reactor.

To insure that the bacteria in the biological reactor maintain viability, it may be necessary to add nutrients to biological reaction. Nitrogen is an important bacteria nutrient for DNA and RNA replication. Nitrogen compounds are preferably to be added to the biological reactor. Phosphate is an important nutrient for biological management of energy; specifically, phosphates assist biological activity in cold temperatures. Phosphate compounds are preferably to be added to the biological reactor. A preferred nutrient for biological reaction would comprise at least one selected from a list consisting of: ammonia, phosphoric acid, ammonium hydroxide, urea, nitrogen salts, phosphate-carbon compounds, nitrogen phosphate salts, nitrogen-carbon compounds, nitrogen-carbon polymers, nitrogen-phosphate-carbon compounds, nitrogen-phosphate-carbon polymers and any combination therein. Nutrients can be added either directly to the biological reactor or to the SS upstream of the biological reactor.

Bacteria are pH sensitive; MOH formation is pH sensitive. It is an embodiment that the SS in biological reaction have a pH between 6.0 and 10.0. It is preferred that the SS in biological reaction have a pH of 9.25+/−0.50. It is an embodiment that the MOHS have a pH between 8.0 and 10.0. It is most preferred that the MOHS have a pH of 9.25+/−0.50. To increase pH, the most preferred base would be any Group I or Group II A metal oxide and/or hydroxide. To increase pH it is preferred to use a base (a base is generally defined as an electron donor). To increase pH, it is preferred to use a polymer, compound or salt containing the OH moiety. To increase pH, it is an embodiment to use a polymer, compound or salt containing an electron donor. The most preferred base comprises at least one selected from the list consisting of: sodium hydroxide, magnesium hydroxide, magnesium oxide, calcium hydroxide, calcium oxide, potassium hydroxide and any combination therein. To decrease pH it is preferred to use an acid. To reduce pH it is an embodiment to use a polymer, compound or salt which is an electron acceptor. To reduce pH it is preferred to use an inorganic acid. To reduce pH it is preferred to use an acid based upon a halogen anion. The most preferred acid is carbonic.

Generally, in the use of oxygen, there are three types of bacteria: anaerobic, facultative and aerobic. Anaerobic strains do not require oxygen, yet are the slowest to remove TOC while creating methane gas and sulfides, along with sulfuric acid. Facultative strains perform at about 3 times the rate of anaerobes and can obtain oxygen either directly or from a salt, such as nitrate, nitrite, sulfate, etc. Aerobic bacteria operate at near 10 times the rate of anaerobes or near 3 times the rate of facultative cultures yet must obtain oxygen directly in order to survive. It is a preferred embodiment to provide an oxygen containing salt to the biological reactor. It is a most preferred embodiment to provide oxygen to the biological reactor; said oxygen can be in the form of pure oxygen, hydrogen peroxide and/or air.

To minimize the size of equipment and maximize the effectiveness of biological reaction, it is an embodiment to use stages of biological reaction, as such it is an embodiment to complete TOC removal in stages of biological reaction, wherein each stage has a lower amount of TOC in the final MOHS. It is an embodiment to reduce TOC in the final MOHS to about less than 100 mg/L. It is a preferred embodiment to reduce the TOC in the final MOHS to about less than 25 mg/L. It is a most preferred embodiment to reduce the TOC in the final MOHS to about less than 5 mg/L. To facilitate TOC removal it is a preferred embodiment to add an easily consumable co-substrate to the biological reactor.

To minimize the size of separation equipment and to maximize the performance of separation equipment, it is preferred to use coagulants and/or flocculants as are known in the art of liquid/solids separation. It is preferred to use coagulants and/or flocculants in any stage of liquid/solids separation of the MOHS and/or the SS. It is an embodiment to use a cationic polymeric coagulant and/or flocculent to separate anionic contaminants and/or bio-solids from MOHS and/or water. It is preferred that said cationic polymeric coagulant and/or flocculant comprise a nitrogen moiety. It is most preferred that said nitrogen moiety be quaternized. It is an embodiment to use an anionic polymeric coagulant and/or flocculant to separate MOH from bacteria and/or water. It is preferred that said anionic polymeric coagulant and/or flocculant comprise acrylamide. It is most preferred that said anionic polymeric coagulant/flocculant comprise an acid based upon acrylate and/or acrylic chemistry.

To create a final cake product, it is preferred that the MOHS from biological reaction and/or from liquid/solids separation, be dried by evaporation of water to form an MOH. It is most preferred that the MOHS from biological reaction and/or from liquid/solids separation be dried by hot air evaporation of water from the MOHS.

To minimize biological activity in the MOHS after biological reaction and/or after liquid/solids separation, it is preferred to add a disinfectant to said MOHS.

It is preferred that the final MOH be used in the production of coagulants. It is most preferred that said MOHS comprise at least one metal selected from a list comprising: aluminum, iron, calcium, magnesium and any combination therein. It is most preferred that the final MOHS mostly comprise aluminum.

To minimize the recycling of pathogens from drinking water and/or wastewater treatment facilities, it is most preferred that any recycled MOHS from a drinking water or wastewater facility, wherein said MOHS is used as a coagulant, have a pH of less than 4.0 for such a period of time as to disinfect solid MOHS of any pathogens. It is preferred that any recycled MOHS from a drinking water and/or a wastewater facility, wherein said MOHS is used in the manufacture of a polynuclear aluminum compound, have a processing history wherein said MOHS is above 100° C. It is most preferred that any recycled MOHS from a drinking water and/or a wastewater facility, wherein said MOHS is used in the manufacture of a polynuclear aluminum compound, have a processing history wherein said MOHS is above 100° C. for a minimum of ½ hour.

### EXAMPLE 1

The City of DeQueen, Ark. operates a municipal drinking water plant using aluminum chlorohydrate as the coagulant. Raw water alkalinity varies from near 10 to near 20 mg/L. Raw Water turbidity varies from near 6 to near 50 NTU with occasional spikes to near 80 NTU. This drinking water plant is of traditional design having a rapid mix, flocculation, clarification and filtration system. Waste aqueous solids, sludge from the clarifiers and filter backwash is sent to an evaporation pond prior to pond overflow into the municipal waste collection system which transports the sludge to the municipal wastewater treatment plant.

A 1 gallon sample of waste aqueous sludge was obtained, chilled and transported prior to testing.

500 ml of the above sludge was placed into a 1 L beaker on a magnetic stir plate. The pH of the sludge was raised to near 9.25 with sodium hydroxide.

1 gram of a dry blend of heterotrophs obtained from Envera, a Waste Water Treatment Product Blend of Lot # 040906, were wetted for 1 hour with an air stone providing a DO concentration of near 3 to 5 mg/L. After 1 hour of wetting and oxygenation, the bacteria liquor was strained through cheesecloth and poured into the 1 L beaker containing the waste sludge. An air stone and a DO Meter were placed into the 1 L beaker. A DO concentration of 2 to 4 mg/L was obtained and maintained for about 6 hours. During the 6 hour period Dissolved Oxygen Uptake Rates (DOURs) were measured at varying intervals obtaining measurements of near 3 to 8 ppm of DO uptake per minute. At near 6 hours of elapsed time the DOUR was tested obtaining near 0.5 DO per minute, thereby demonstrating that nearly all of the consumable substrate (TOC) had been consumed by the heterotrophs.

The MOHS from above was then poured into a second 1 L beaker, wherein near 25 ppm of CV 3650 (DADMAC 20% active having a viscosity of near 1800 cps) was added and mixed at high speed for near 30 seconds and then mixed at slow speed for near 5 minutes. A brownish floc developed and settled from a grayish liquid. The grayish liquid was then decanted into a third beaker, wherein about 5 ppm of CV 6130 (anionic polyacrylamide emulsion which is 40% active and 30% anionic wherein the anionic monomer is based upon an acrylic acid and the molecular weight is near 8 million, as measured by intrinsic viscosity) was added and mixed at high speed for near 30 seconds and then mixed at slow speed for near 5 minutes. A grayish floc developed and settled from a near clear water.

### EXAMPLE 2

The City of Nashville, Ark. operates a municipal drinking water plant using CV 1766 (a blend of aluminum chlorohydrate and CV 3650) as the coagulant. Raw water alkalinity varies from near 10 to near 30 mg/L. Raw Water turbidity varies from near 6 to near 40 NTU with occasional spikes to near 60 NTU. This drinking water plant is of traditional design having a rapid mix, flocculation, clarification and filtration system. Waste aqueous solids, sludge from the clarifiers and filter backwash is wasted to an evaporation pond.

A 1 gallon sample of waste aqueous sludge was obtained, chilled and transported prior to testing.

500 ml of the above sludge was placed into a 1 L beaker on a magnetic stir plate. The pH of the sludge was raised to near 9.25 with sodium hydroxide.

1 gram of a dry blend of heterotrophs obtained from Envera, a Waste Water Treatment Product Blend of Lot # 040906, were wetted for 1 hour with an air stone providing a DO concentration of near 3 to 5 mg/L. After 1 hour of wetting and oxygenation, the bacteria liquor was strained through cheesecloth and poured into the 1 L beaker containing the waste sludge. An air stone and a DO Meter were placed into the 1 L beaker. A DO concentration of 2 to 4 mg/L was obtained and maintained for about 6 hours. During the 6 hour period Dissolved Oxygen Uptake Rates (DOURs) were measured at varying intervals obtaining measurements of near 3 to 8 ppm of DO uptake per minute. At near 6 hours of elapsed time the DOUR was tested obtaining near 0.8 DO per minute, thereby demonstrating that nearly all of the consumable substrate (TOC) had been consumed by the heterotrophs.

The MOHS from above was then poured into a second 1 L beaker, wherein near 5 ppm of CV 6140 (a Q9 cationic polyacrylamide emulsion 40% active having a 40% cationic charge and a molecular weight of near 10 million, as measured by intrinsic viscosity) was added and mixed at high speed for near 30 seconds and then mixed at slow speed for near 5 minutes. A brownish floc developed and settled from a grayish liquid. The grayish liquid was then decanted into a third beaker, wherein about 5 ppm of CV 6130 was added and mixed at high speed for near 30 seconds and then mixed at slow speed for near 5 minutes. A grayish floc developed and settled from a near clear water.

### EXAMPLE 3

A sample of aluminum chloride, GPAC 2200 from Gulbrandsen Company, Inc., was placed into a 1 L beaker. The pH of the sample was raised to near 8.75 with sodium hydroxide creating a grayish/reddish liquid. Into the beaker was then placed near 1.5 ppm CV 6130 was added and mixed at high speed for near 30 seconds and then mixed at slow speed for near 5 minutes. A grayish/reddish floc developed and settled from a near clear water.

### EXAMPLE 4

A sample of aluminum chlorohydrate, GPAC 850 from Gulbrandsen Company, Inc., was placed into a 1 L beaker. The pH of the sample was raised to near 9.0 with lime (CaO) creating a grayish liquid containing many solids. Into the beaker was then placed near 5 ppm CV 6130 was added and mixed at high speed for near 30 seconds and then mixed at slow speed for near 5 minutes. A grayish floc developed and settled from a near clear water.

### EXAMPLE 5

A sample of ferric sulfate from Pioneer Chemical Company was placed into a 1 L beaker. The pH of the sample was “carefully” raised to near 9.50 with 25% hydrogen peroxide creating an orange/grayish liquid. Into the beaker was then placed near 2 ppm of CV 6130 was added and mixed at high speed for near 30 seconds and then mixed at slow speed for near 5 minutes. An orange/grayish floc developed and settled from a near clear water.

Certain objects are set forth above and made apparent from the foregoing description. However, since certain changes may be made in the above description without departing form the scope and/or the intent of the invention, it is intended that all matters contained in the foregoing description shall be interpreted as illustrative only of the principals of the invention and not in a limiting sense. With respect to the above description, it is to be realized that any descriptions, drawings and examples deemed readily apparent and/or obvious to one of skill in the art and all equivalent relationships to those described in the specification are intended to be encompassed by the present invention.

Further, since numerous modifications and changes will readily occur to those skilled in the art, it is not desired to limit the invention to the exact construction and operation shown and described, and accordingly, all suitable modifications and equivalents may be resorted to, falling within the scope of the invention. It is also to be understood that the following claims are intended to cover all of the generic and specific features of the invention herein described, and all statements of he scope of the invention, which, as a matter of language, might be said to fall in between.

1. A method of recovering metal(s) from an aqueous solution, wherein
said aqueous solution flows into a biological reactor, wherein
said biological reactor comprise bacteria which remove TOC from said aqueous solution and wherein
said aqueous solution flows from said biological reactor to liquid/solid separation, wherein
said metal(s) are mostly separated from said solution.

2. The method of claim 1, wherein
the pH of said aqueous solution is increased and/or decreased to obtain a pH of approximately between 8.0 and 10.0.

3. The method of claim 1, wherein
the pH of said aqueous solution is increased and/or decreased to near 9.15+/−0.5.

4. The method of claim 1, wherein said aqueous solution is waste sludge from at least one drinking water and/or one wastewater purification facility.

5. The method of claim 1, wherein said aqueous solution comprises a catalyst.

6. The method of claim 1, wherein said metal(s) comprise at least one selected from a list consisting of: aluminum, iron, calcium, magnesium and any combination therein.

7. The method of claim 1, wherein said metal(s) is mostly aluminum.

8. The method of claim 1, wherein said biological reaction comprises fermentation-raised bacteria.

9. The method of claim 1, wherein said biological reaction comprises non-pathogenic bacteria.

10. The method of claim 1, wherein said biological reaction comprises selectively cultured bacteria.

11. The method of claim 1, wherein said biological reaction comprises at least one biological culture selected from a list consisting of: Acinobacter, Nitrobacter, Enterobacter, Thiobacillus and Thiobacillus Denitrificanus, Psudomonas, Escherichia, Artobacter, Achromobacter, Bdellovibrio, Thiobacterium, Macromonas, Bacillus, Cornebacterium, Aeromonas, Alcaligenes, Falvobacterium, Vibro, fungi and any combination therein.

12. The method of claim 1, wherein said biological reaction is messophilic and/or said biological reaction comprises messophilic bacteria.

13. The method of claim 1, wherein said biological reaction is thermophilic and/or said biological reaction comprises thermophilic bacteria.

14. The method of claim 1, wherein said biological reaction comprises psychrophiles.

15. The method of claim 1, wherein said biological reaction comprises psychotrophs.

16. The method of claim 1, wherein a nutrient(s) is added to biological reaction.

17. The method of claim 16, wherein said nutrient(s) comprise at least one selected from a list consisting of: ammonia, phosphoric acid, ammonium hydroxide, urea, nitrogen salts, phosphate-carbon compounds, nitrogen phosphate salts, nitrogen-carbon compounds, nitrogen-carbon polymers, nitrogen-phosphate-carbon compounds, nitrogen-phosphate-carbon polymers and any combination therein.

18. The method of claim 1, wherein said pH is increased by the addition of a base.

19. The method of claim 18, wherein said base comprises a polymer, compound or salt which is an electron donor.

20. The method of claim 19, wherein said base comprises a Group I or Group II A metal oxide and/or hydroxide.

21. The method of claim 20, wherein said base comprises at least one selected from the list consisting of: sodium hydroxide, magnesium hydroxide, magnesium oxide, calcium hydroxide, calcium oxide, potassium hydroxide and any combination therein.

22. The method of claim 19, wherein said base comprises a polymer, compound or salt which contains an OH moiety.

23. The method of claim 1, wherein said pH is reduced by the addition of an acid.

24. The method of claims 23, wherein said acid comprises a salt, compound or polymer which an electron acceptor.

25. The method of claim 24, wherein said acid comprises a halogen anion.

26. The method of claim 24, wherein said acid comprises an inorganic salt anion.

27. The method of claim 24, wherein said acid comprises carbonic acid.

28. The method of claim 1, wherein said liquid/solids separation is performed by coagulating said bacteria from said biological reactor with a cationic organic polymer.

29. The method of claim 28, wherein said cationic polymer comprises a cationic nitrogen moiety.

30. The method of claim 29, wherein said nitrogen moiety is quaternized.

31. The method of claim 29, wherein said cationic polymer comprises acrylamide.

32. The method of claim 1, wherein said liquid/solids separation is performed by coagulating said metals in a metal oxide and/or metal hydroxide form(s) with an anionic polymer.

33. The method of claim 32, wherein said anionic polymer comprises an acidified moiety based upon an acrylate and/or an acrylic moiety.

34. The method of claim 32, wherein said anionic polymer comprises acrylamide.

35. The method of claim 1, wherein said metal(s) are recycled to manufacture a coagulant.

36. The method of claim 35, wherein said coagulant is in a salt form having a pH of less than approximately 4.0.

37. The method of claim 35, wherein said coagulant is in the form of a polynuclear aluminum compound, and wherein
said polynuclear aluminum compound have a temperature history of greater than 100° C.

38. The method of claim 1, wherein after biological reaction
said aqueous solution comprises a disinfectant.

39. The method of claim 1, wherein said bacteria from said biological reactor are treated per the US EPA 503 Regulations.

40. The method of claim 39, wherein said bacteria are digested in a thermophilic digester.
